SEQUENCE LISTING (22 sequences)

SEQ ID NO: 1 - protein, 453 aa, human
MASPSLPGSDCSQIIDHSHVPEFEVATWIKITLILVYLIIFVMGLLGNSATIRVTQVLQKKGYLQKEVTDHMVSLACSDILVFLIGMPMEFYSIIWNPLTTSSYTLSCKLHTFLFEACSYATLLHVLTLSFERYIAICHPFRYKAVSGPCQVKLLIGFVWVTSALVALPLLFAMGTEYPLVNVPSHRGLTCNRSSTRHHEQPETSNMSICTNLSSRWTVFQSSIFGAFVVYLVVLLSVAFMCWNMMQVLMKSQKGSLAGGTRPPQLRKSESEESRTARRQTIIFLRLIVVTLAVCWMPNQIRRIMAAAKPKHDWTRSYFRAYMILLPFSETFFYLSSVINPLLYTVSSQQFRRVFVQVLCCRLSLQHANHEKRLRVHAHSTTDSARFVQRPLLFASRRQSSARRTEKIFLSTFQSEAEPQSKSQSLSLESLEPNSGAKPANSAAENGFQEHEV

SEQ ID NO: 2 - DNA, 1362 bp, human
atggcttcacccagcctcccgggcagtgactgctcccaaatcattgatcacagtcatgtccccgagtttgaggtggccacctggatcaaaatcacccttattctggtgtacctgatcatcttcgtgatgggccttctggggaacagcgccaccattcgggtcacccaggtgctgcagaagaaaggatacttgcagaaggaggtgacagaccacatggtgagtttggcttgctcggacatcttggtgttcctcatcggcatgcccatggagttctacagcatcatctggaatcccctgaccacgtccagctacaccctgtcctgcaagctgcacactttcctcttcgaggcctgcagctacgctacgctgctgcacgtgctgacactcagctttgagcgctacatcgccatctgtcaccccttcaggtacaaggctgtgtcgggaccttgccaggtgaagctgctgattggcttcgtctgggtcacctccgccctggtggcactgcccttgctgtttgccatgggtactgagtaccccctggtgaacgtgcccagccaccggggtctcacttgcaaccgctccagcacccgccaccacgagcagcccgagacctccaatatgtccatctgtaccaacctctccagccgctggaccgtgttccagtccagcatcttcggcgccttcgtggtctacctcgtggtcctgctctccgtagccttcatgtgctggaacatgatgcaggtgctcatgaaaagccagaagggctcgctggccgggggcacgcggcctccgcagctgaggaagtccgagagcgaagagagcaggaccgccaggaggcagaccatcatcttcctgaggctgattgttgtgacattggccgtatgctggatgcccaaccagattcggaggatcatggctgcggccaaacccaagcacgactggacgaggtcctacttccgggcgtacatgatcctcctccccttctcggagacgtttttctacctcagctcggtcatcaacccgctcctgtacacggtgtcctcgcagcagtttcggcgggtgttcgtgcaggtgctgtgctgccgcctgtcgctgcagcacgccaaccacgagaagcgcctgcgcgtacatgcgcactccaccaccgacagcgcccgctttgtgcagcgcccgttgctcttcgcgtcccggcgccagtcctctgcaaggagaactgagaagattttcttaagcacttttcagagcgaggccgagccccagtctaagtcccagtcattgagtctcgagtcactagagcccaactcaggcgcgaaaccagccaattctgctgcagagaatggttttcaggagcatgaagtttga

SEQ ID NO: 3 - protein, 24 aa, human
FNAPFDVGIKLSGVQYQQHSQALG

SEQ ID NO: 4 - protein, 23 aa, human
FNAPFDVGIKLSGVQYQQHSQAL

SEQ ID NO: 5 - protein, 24 aa, sheep
FNAPFNIGIKLSGAQSLQHGQTLG

SEQ ID NO: 6 - protein, 23 aa, sheep
FNAPFNIGIKLSGAQSLQHGQTL

SEQ ID NO: 7 - protein, 24 aa, cow
FNAPFNIGIKLAGAQSLQHGQTKG

SEQ ID NO: 8 - protein, 23 aa, cow
FNAPFNIGIKLAGAQSLQHGQTK

SEQ ID NO: 9 - protein, 24 aa, monkey
FNAPFDVGIKLSGVQYQQHSQALG

SEQ ID NO: 10 - protein, 23 aa, monkey
FNAPFDVGIKLSGVQYQQHSQAL

SEQ ID NO: 11 - protein, 24 aa, mouse
FNAPFDVGIKLSGAQYQQHGRALG

SEQ ID NO: 12 - protein, 23 aa, mouse
FNAPFDVGIKLSGAQYQQHGRAL

SEQ ID NO: 13 - protein, 24 aa, rat
FNAPFDVGIKLSGAQYQQHGRALG

SEQ ID NO: 14 - protein, 23 aa, rat
FNAPFDVGIKLSGAQYQQHGRAL

SEQ ID NO: 15 - protein, 24 aa, gerbil
FNAPFDVGIKLSGAQYQQHGRALG

SEQ ID NO: 16 - protein, 23 aa, gerbil
FNAPFDVGIKLSGAQYQQHGRAL

SEQ ID NO: 17 - protein, 24 aa, pig
FNAPCDVGIKLSGAQSDQHGQPLG

SEQ ID NO: 18 - protein, 23 aa, pig
FNAPCDVGIKLSGAQSDQHGQPL

SEQ ID NO: 19 - protein, 24 aa, dog
FNAPFDVGIKLSGPQYHQHGQALG

SEQ ID NO: 20 - protein, 23 aa, dog
FNAPFDVGIKLSGPQYHQHGQAL

SEQ ID NO: 21 - protein, 23 aa, human
FNAPFDVGIKLSGVQYQQHSQAL

SEQ ID NO: 22 - protein, 24 aa, human
FNAPFDVGIKLSGVLYQQHSQALG

(19) United States
(12) Patent Application Publication
(10) Pub. No.: US 2007/0042409 A1
(43) Pub. Date: Feb. 22, 2007

(54) Mammalian obestatin receptors

(76) Inventors: Aaron J.W. Hsueh, Stanford, CA (US); Jian V. Zhang, Mountain View, CA (US); Ching-Wei Luo, Menlo Park, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/499,030

(22) Filed: Aug. 4, 2006

Related U.S. Application Data
(60) Provisional application No. 60/705,796, filed on Aug. 5, 2005.

(51) Int. Cl. C12Q 1/68; G01N 33/53; A61K 38/16

(52) U.S. Cl. 435/6; 435/7.1; 514/2

(57) ABSTRACT

A high affinity obestatin receptor is provided; the orphan receptor GPR39. The receptor mediates obestatin activities. The obestatin receptor (GPR39) and fragments thereof, particularly soluble fragments thereof, are useful as therapeutic agents capable of inhibiting the action of obestatin. In addition to use as a therapeutic agent, GPR39 polypeptides are utilized in screening and research methods for the determination of specific analogs, agonists, antagonist mimetics and agents that modulate production, metabolism, and disposition of GPR39 activities. Conditions treatable with GPR39 agonists or antagonists include regulation of weight, blood pressure and heart rate, and gastric empyting.

### BACKGROUND OF THE INVENTION

Polypeptide hormones and their receptors play important roles in the maintenance of homeostasis in multicellular organisms, including, for example, maintenance of weight. Recent sequencing of the genomes of human and several animal models provide an unprecedented opportunity to identify novel polypeptide ligands based on sequence homology among paralogous ligand genes. In addition, a large number of putative G protein-coupled receptors without known ligands have been predicted based on their characteristic seven transmembrane domains. Although the ligands for some of these ‘orphan’ GPCRs have been identified based on biochemical purification and other approaches, the ligands for many of them are still unknown. There is considerable interest for clinical and research purposes in the discovery and development of agents that act on these receptors.

Small synthetic molecules called growth hormone secretagogues (GHSs) stimulate the release of growth hormone from the pituitary. They act through the growth hormone secretagogue receptor, a G protein-coupled receptor. An endogenous ligand specific for GHSR was reported by Kojima et al. (1999) Nature 402: 656-660. The ligand is a peptide of 28 amino acids in which the serine-3 residue is n-octanoylated. The acylated peptide specifically releases growth hormone both in vivo and in vitro, and O-n-octanoylation at serine-3 is essential for the activity. The GH-releasing peptide was termed “ghrelin”. Human ghrelin has substantial sequence identity to rat ghrelin, differing by only 2 amino acids. The occurrence of ghrelin in both rat and human indicates that GH release from the pituitary may be regulated not only by hypothalamic growth hormone-releasing hormone, but also by ghrelin. Ghrelin is expressed in the stomach, apparently by endocrine cells. Ghrelin is also expressed in neurons, e.g. in the hypothalamic arcuate nucleus.

Ghrelin is the only known circulating orexigen important in the control of energy balance and body weight. In addition to the observed preprandial rise in plasma ghrelin levels in man, in vivo treatment with ghrelin led to increases in feeding and body weight in rodents. Ghrelin has also been found to stimulate pituitary GH release, to regulate gastric motility and acid secretion, as well as to modulate the cardiovascular system, pancreatic exocrine and endocrine functions, reproductive axis, sleep, and behavior.

Ghrelin is one of the brain/gut peptide hormones that are usually derived from large preproproteins following proteolytic cleavages and other posttranslational modifications. Human ghrelin, a 28 amino acid peptide, is derived from a prepropeptide of 117 residues. Contrary to the orexigenic effects of ghrelin, a novel amidated peptide derived from proghrelin, termed obestatin (also referred to as GLAP, or ghrelin-associated peptide) suppresses food intake, inhibits jejunum contraction, and decreases body weight gain. Unlike ghrelin, serum levels of obestatin are not regulated by fasting or refeeding. Radioligand binding assays indicate that obestatin interacts with high affinity binding sites in gastrointestinal, pituitary, and other tissues.

Dixit et al. (2004) J. Clin. Invest. 114: 57-66, 2004 demonstrated that ghrelin and its receptor, GHSR, are expressed in human T lymphocytes and monocytes, where ghrelin acts via GHSR to inhibit specifically the expression of proinflammatory anorectic cytokines such as IL1-beta, IL6, and TNF-alpha. Ghrelin led to a dose-dependent inhibition of leptin-induced cytokine expression, whereas leptin upregulated GHSR expression on human T lymphocytes. Dixit et al. (2004) proposed the existence of a reciprocal regulatory network by which ghrelin and leptin control immune cell activation and inflammation. In a murine model of endotoxemia, Dixit et al. (2004) also showed that ghrelin has potent antiinflammatory effects and attenuates endotoxin-induced anorexia.

Orphan receptors related to the human orphan growth factor secretogue receptor (GHS-R) are described by McKee et al., (1997) Genomics 46(3):426-34; Smith et al., (1999) Horm Res 51 Suppl 3:1-8; Palyha et al. (2000) Mol. Endo. 14:160-169. Holst et al., (2004) JBC 279:53806-53817 describe the basis of constitutive activity of the ghrelin receptor family.

### SUMMARY OF THE INVENTION

A high affinity obestatin receptor is provided; the orphan receptor GPR39. The receptor mediates obestatin activities of, for example, regulation of gastric activity, jejunal movement, blood pressure and weight. The GPR39 receptor is not activated by ghrelin, which is derived from the same gene as obestatin, but which undergoes differential posttranslational processing and modification and has antagonistic action to obestatin in body weight regulation and other activities.

The obestatin receptor (GPR39) and fragments thereof, particularly soluble fragments thereof, are useful as therapeutic agents capable of inhibiting the action of obestatin. In addition to use as a therapeutic agent, GPR39 polypeptides are utilized in screening and research methods for the determination of specific analogs, agonists, antagonist mimetics and agents that modulate production, metabolism, and disposition of GPR39 activities. Conditions treatable with GPR39 agonists or antagonists include regulation of weight, blood pressure and heart rate, and gastric empyting. In one embodiment of the invention, obestatin receptor, and agonists and antagonists thereof find use where it is desirable to regulate blood pressure. In another embodiment, the compositions find use where weight regulation is desirable.

### BRIEF DESCRIPTION OF THE FIGURES

FIG. 1 Bioinformatic prediction of conserved obestatin. Amino acid sequence of pre-proghrelin from diverse mammalian species denoting the signal peptide (italicized), mature ghrelin (shaded), and the flanking obestatin (underlined). Consensus basic residues representing putative convertase cleavage sites are shown as white letters in a black background.

FIG. 2 Characterization of endogenous obestatin. a) Competition of I¹²⁵-obestatin binding to obestatin antibodies by tissue extracts. I¹²⁵-obestatin was incubated with obestatin antibodies with or without different dilutions of tissue extracts and the obestatin standard. b) Gel permeation chromatography of obestatin in stomach extracts. Pooled stomach tissues from 30 rats were extracted and eluted from a Sep-Pak C-18 column before loading onto a Sephadex G-50 column. The column was calibrated with blue dextran (V₀), cytochrome c (CC), and potassium chromate (Vₜ). Peak 1 detected by the obestatin antibodies represented the putative obestatin peptide and peak 2 represented a obestatin fragment. c) Ion exchange FPLC analysis of peak 1 fractions monitored by the obestatin immunoassay. d) Peptide mapping using mass spectrometry and the predicted amino acid sequence of obestatin. e) Serum levels of ghrelin and obestatin during fasting and re-feeding. Adult male rats were fasted for two days. Following fasting, some animals were allowed access to food, dextrose solution or water for 2 h before serum hormone determination using specific radioimmunoassays.

FIG. 3 Regulation of gastrointestinal functions by obestatin. a) Suppression of cumulative food intake following treatment with different doses of obestatin, the non-amidated obestatin (NA-obestatin), and/or ghrelin. Mice injected with urocortin served as positive controls. b) Suppression of gastric emptying activity by obestatin. c) Treatment with obestatin suppressed body weight gain induced by ghrelin. d-f) Treatment with obestatin suppressed the contractile activity of jejunum muscle strips and the stimulatory effect of ghrelin. Representative tracing (d), percentage of maximal responses (e) and release of growth factor (f) are shown.

FIG. 4 obestatin activates the orphan receptor GPR39. a) High affinity binding of I¹²⁵-obestatin to CHO cells overexpressing GPR39. Saturation and Scatchard plots are shown. b) Hormonal specificity of I¹²⁵-obestatin binding to GPR39. c). obestatin activation of cAMP production. d). Ghrelin, but not obestatin, stimulated cAMP production in cells transfected with GHSR and the chimeric Gsq protein. e). obestatin activation of the SRE-luciferase reporter. f). Real-time RT-PCR analyses of GPR39 transcript levels in diverse tissues.

FIG. 5. Sequence details at position 50 (boxed) for human (SEQ ID NO:23); chimp (SEQ ID NO:24); rat (SEQ ID NO:25), mouse (SEQ ID NO:26), dog (SEQ ID NO:27) and cattle (SEQ ID NO:28), demonstrating the conservation of the valine at this position.

FIGS. 6A and 6B. A comparison of the binding affinity of human obestatin receptor having an alanine (A) or a valine (B) at position 50.

### DESCRIPTION OF THE SPECIFIC EMBODIMENTS

GPR39 is identified as the receptor for the peptide hormone obestatin. The receptor mediates obestatin activities, including regulating blood pressure and weight. GPR39 and fragments thereof, particularly soluble fragments thereof, are useful as therapeutic agents capable of inhibiting the action of obestatin. In addition to use as a therapeutic agent, GPR39 polypeptides are utilized in screening and research methods for the determination of specific analogs, agonists, antagonist mimetics and agents that modulate production, metabolism, and disposition of obestatin activities.

The regulatory peptide obestatin plays an important role in maintaining homeostasis of gastrointestinal, cardiovascular, hypothalamus-pituitary axis, and the central nervous system. In one embodiment of the invention, modulators of GPR39 activity are used in the treatment of obesity. In another embodiment, modulators of GPR39 activity are used in the modulation of cardiovascular function, including heart rate and blood pressure regulation.

### GPR39 Compositions

As used herein, the term GPR39 refers to the polypeptide set forth as SEQ ID NO:1, homologs, orthologs, variants and fragments thereof. It is expressed in the stomach, the small intestine, and in the central nervous system. Human GPR39 is a polypeptide of 453 amino acids in length, and is encoded by the polynucleotide sequence of SEQ ID NO:2. A variant of GPR39 comprises the sequence set forth in SEQ ID NO:1 or a fragment thereof having a substitution of valine for alanine at position 50.

GPR39 peptides, which can be used in the methods of the invention, comprise at least about 10 amino acids, usually at least about 12 amino acids, at least about 15 amino acids, and which may include up to or more than 50 amino acids of a GPR39 peptide, including domains and larger fragments of about 100 amino acids or more; and modifications thereof, and may further include fusion polypeptides as known in the art in addition to the provided sequences. A combination of one or more forms may be used. The GPR39 sequence may be from any mammalian or avian species, e.g. primate sp., particularly humans; rodents, including mice, rats and hamsters; rabbits; equines, bovines, canines, felines; etc. Of particular interest are the human proteins.

Functional variants of the GPR39 polypeptide are of interest. Such variants may have substantial sequence similarity to a native GPR39 sequence, for example SEQ ID NO:1, usually at least about 90% sequence identity; at least about 95% sequence identity; up to at least about 99% sequence identity or more. Such variants may comprise 1, 2, 3, 4, 5, or more amino acid substitutions, deletions or additions, including conservative substitutions. Such functional variants may be defined as binding at high affinity, usually at least about 10 nM Kd, or at least about 5 nM Kd, to a obestatin (obestatin) peptide, where the obestatin peptide may include, without limitation, the native amidated obestatin peptides (SEQ ID NO:3) FNAPFDVGIKLSGVQYQQHSQALG (human); (SEQ ID NO:4) FNAPFDVGIKLSGVQYQQHSQAL-NH2 (human); (SEQ ID NO:21) FNAPFDVGIKLSGVLYQQHSQALG (human); (SEQ ID NO:22) FNAPFDVGIKLSGVLYQQHSQAL-NH2 (human); (SEQ ID NO:5) FNAPFNIGIKLSGAQSLQHGQTLG (sheep); (SEQ ID NO:6) FNAPFNIGIKLSGAQSLQHGQTL-NH2 (sheep); (SEQ ID NO:7) FNAPFNIGIKLAGAQSLQHGQTKG (bos); (SEQ ID NO:8) FNAPFNIGIKLAGAQSLQHGQTK-NH2 (bos); (SEQ ID NO:9) FNAPFDVGIKLSGVQYQQHSQALG (macaca); (SEQ ID NO:10) FNAPFDVGIKLSGVQYQQHSQAL-NH2 (macaca); (SEQ ID NO:11) FNAPFDVGIKLSGAQYQQHGRALG (mouse); (SEQ ID NO:12) FNAPFDVGIKLSGAQYQQHGRAL-NH2 (mouse); (SEQ ID NO:13) FNAPFDVGIKLSGAQYQQHGRALG (rat); (SEQ ID NO:14) FNAPFDVGIKLSGAQYQQHGRAL-NH2 (rat); (SEQ ID NO:15) FNAPFDVGIKLSGAQYQQHGRALG (gerbil); (SEQ ID NO:16) FNAPFDVGIKLSGAQYQQHGRAL-NH2 (gerbil); (SEQ ID NO:17) FNAPCDVGIKLSGAQSDQHGQPLG (pig); (SEQ ID NO:18) FNAPCDVGIKLSGAQSDQHGQPL-NH2 (pig); (SEQ ID NO:19) FNAPFDVGIKLSGPQYHQHGQALG (dog); (SEQ ID NO:20) FNAPFDVGIKLSGPQYHQHGQAL-NH2 (dog).

Functional variants may also be assessed by the ability of a variant to activate pathways mediated by the wild-type GPR39 polypeptide, for example where the variant has an activity at least equal to the wild-type protein; and activity greater than the wild-type protein; or an activity not less than about 25% the activity of the wild-type protein. The activity may be ligand dependent or ligand independent, usually ligand dependent.

GPR39 has been identified as having certain constitutive activities (i.e. ligand independent) by Hoist et al., supra. (herein specifically incorporated by reference) in several different pathways. GPR39 was found to have a lower level of constitutive G_{q}/phospholipase C signaling compared to the ghrelin receptor, but higher constitutive activity through the steroid responsive element (SRE) pathway. With respect to CRE-mediated transcription, the ghrelin receptor showed very strong ligand-independent activity, reaching a level 2-fold higher than the neurotensin receptor, whereas GPR39 showed only a very limited degree of constitutive activity in this signaling pathway. GPR39 was also silent with respect to ligand-independent signaling through ERK1/2 phosphorylation. GPR39 was also reported not to internalize in the absence of the ligand. A common structural “volume knob” or lever was proposed for this family of receptors in the form of an aromatic cluster of residues on the inner face of TMs VI and VII. Variants of interest for the methods of the present invention optionally include modifications in these regions.

The receptors of the invention have a number of important physiological functions, including modulation of body weight and metabolism, and modulation of cardiovascular activity. As used herein, GPR39 directed agents, which include GPR39 peptides and fragments thereof, specific analogs, agonists, antagonist mimetics and agents that modulate production, metabolism, and disposition of GPR39 activities, including anti-sense reagents, RNAi, coding sequences, etc. Modulators of cardiovascular activity refer to molecules that alter the physiological function of the cardiovascular system, including, without limitation, the blood pressure and heart rate, etc. Modulators of weight affect the intake of food, gastric motility, energy balance, weight homeostasis, etc. Such modulators include agonists that enhance, potentiate and/or mimic the activity of an obestatin peptide; and antagonists, which inhibit or decrease the activity of an obestatin peptide.

### Compound Screening

The availability of purified GPR39 and other components in the signaling pathways, e.g. obestatin, etc., allows in vitro reconstruction of the signaling pathway. Two or more of the components may be combined in vitro, and the behavior assessed in terms of production of cAMP; SRE mediated signaling; phospholipase C signaling; etc. The components may be modified by sequence deletion, substitution, etc. to determine the functional role of specific residues. The screening may be performed in vitro or in vivo. Preferred methods include the use of obestatin as a reference for activity, and may include primary or secondary screening of animal models where the effect of a candidate agent on obesity, regulation of blood pressure, and the like are monitored.

Drug screening identifies agents that mimic GPR39 activity, either as an antagonist or as an agonist. A wide variety of assays may be used for this purpose, including labeled in vitro protein-protein binding assays, electrophoretic mobility shift assays, immunoassays for protein binding, and the like. Areas of investigation include the development of treatments for obesity, for regulation of blood pressure, for regulating intestinal motility; and the like.

The term “agent” as used herein describes any molecule, e.g. protein or pharmaceutical, e.g. small organic molecules, with the capability of altering or mimicking the physiological function of GPR39. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Candidate agents of interest for screening also include peptide agents, including, without limitation, derivatives, analogs and fragments of obestatin, for example truncated versions of obestatin, particularly comprising deletions of the first 1, 2, 3, 4, 10, etc. residues. Generally, peptide agents encompassed by the methods provided herein range in size from about 3 amino acids to about 100 amino acids, with peptides ranging from about 3 to about 25 being typical and with from about 3 to about 12 being more typical. Peptide agents can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. In addition, such peptides can be produced by translation from a vector having a nucleic acid sequence encoding the peptide using methods known in the art (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein).

Peptide libraries can be constructed from natural or synthetic amino acids. For example, a population of synthetic peptides representing all possible amino acid sequences of length N (where N is a positive integer), or a subset of all possible sequences, can comprise the peptide library. Nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methy1 amino acids, C α-methyl amino acids, N α-methyl amino acids, and amino acid analogs in general. Furthermore, the amino acid can be D (dextrorotary) or L (levorotary).

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Obestatin, e.g. the peptide set forth in any one of SEQ ID NO:3-22 or analogs thereof may be useful in the screening assays, as competitors, controls, in structural studies of binding sites, etc.

### Cell-Free Assays

Cell-free assay methods generally comprise: a) contacting a test agent with a sample containing a GPR39 polypeptide; and b) assaying an activity of the polypeptide in the presence of the substance, e.g. binding, conformational changes, etc. An increase or a decrease in the measured activity or binding in comparison to the activity in a suitable control (e.g., a sample comprising a polypeptide in the absence of the substance being tested) is an indication that the substance modulates an activity of the polypeptide.

The above screening methods may be designed a number of different ways, where a variety of assay configurations and protocols may be employed, as are known in the art. For example, one of the components may be bound to a solid support, or provided in a lipid bilayer and the remaining components contacted with the bound or membrane-bound component. Such assays may include the use of obestatin or an analog thereof as a competitor or control.

The components of the method may be combined at substantially the same time or at different times. Incubations are performed at any suitable temperature, typically between 40 and 40° C. Incubation periods are selected for optimum activity, but may also be optimized to facilitate rapid high-throughput screening. Typically between 0.1 and 1 hours will be sufficient. Following the contact and incubation steps, the subject methods will generally, though not necessarily, further include a washing step to remove unbound components, where such a washing step is generally employed when required to remove label that would give rise to a background signal during detection, such as radioactive or fluorescently labeled non-specifically bound components.

### Cell-Based Assays

Cell-based assay generally involve contacting a cell expressing GPR39 with a test agent, and determining the effect, if any, on an activity of the cell. In some embodiments, cells comprising a mutated GPR39 gene are used. The accumulation of IP, changes in cAMP, etc. may be measured, and may utilize an obestatin sequence as a control, competitor, etc. For example, a cell of the gut or nervous system that expresses GPR39 may be used, or a cell in which a construct for GPR39 expression has been introduced. The cell is contacted with a candidate agent, and the resulting ligand specific activity measured, and compared to a control cell in the absence or presence of a GPR39 ligand.

It should be understood that in the drug screening and protein modification assays provided herein, a number of iterative cycles of any or all of the steps may be performed to optimize the selection. For example, assays and drug screens that monitor activity in the presence and/or absence of a known ligand are also included in the present invention and can be employed as an assay or drug screen, usually as a single step in a multi-step protocol.

Once a potential modulator/inhibitor is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential modulator may be synthesized de novo. The de novo synthesis of one or even a relatively small group of specific compounds is reasonable in the art of drug design.

The success of both database and de novo methods in identifying compounds with activities similar to the compound of interest depends on the identification of the functionally relevant portion of the compound of interest. For drugs, the functionally relevant portion may be referred to as a pharmacophore, i.e. an arrangement of structural features and functional groups important for biological activity. Not all identified compounds having the desired pharmacophore will act as a modulator of inflammation. The actual activity can be finally determined only by measuring the activity of the compound in relevant biological assays. However, the methods of the invention are extremely valuable because they can be used to greatly reduce the number of compounds that must be tested to identify an actual therapeutic agent.

In order to determine the biological activity of a candidate pharmacophore it is preferable to measure biological activity at several concentrations of candidate compound. The activity at a given concentration of candidate compound can be tested in a number of ways.

### Antibodies Specific for GPR39 Polypeptides

The present invention may utilize antibodies specific for GPR39 polypeptides, e.g. any one of the variants, polypeptides, or domains described above, as a GPR39 directed agent. Such antibodies are useful, for example, as an antagonist or agonist of GPR39, depending on whether the antibody is blocking or activating.

The GPR39 polypeptides of the invention are useful for the production of antibodies, where short fragments provide for antibodies specific for the particular polypeptide, and larger fragments or the entire protein allow for the production of antibodies over the surface of the polypeptide. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a GPR39 polypeptide, particularly a human GPR39 polypeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### Formulations

The compounds of this invention can be incorporated into a variety of formulations for therapeutic administration. Particularly, agents specifically bind to and activate GPR39; agents that block binding of native ligands, e.g. obestatin, agents that modulate expression of GPR39; GPR39 polypeptides and analogs or fragments thereof; etc., are formulated for administration to patients for various clinical purposes, as described herein. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The agents may be systemic after administration or may be localized by the use of an implant that acts to retain the active dose at the site of implantation.

In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the targeted site, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic lipids, such as phosphatidylcholine. The remaining lipid will normally be neutral lipids, such as cholesterol, phosphatidyl serine, phosphatidyl glycerol, and the like.

### Uses of GPR39

In light of the pharmacologic activities of obestatin, numerous clinical indications are evident for the use of pharmaceutical compositions containing GPR39 directed agents as described above. For example, clinical indications for such compositions may find use include treatment of obesity, as a cardioprotective agent, and as a hypotensive agent.

Human obesity is a widespread and serious disorder, affecting a high percentage of the adult population in developed countries. In spite of an association with heart disease, type II diabetes, cancer, and other conditions, few persons are able to permanently achieve significant weight loss. The subject peptides are administered to obese patients for purposes of appetite suppression. Patients may use various criteria for determining obesity. Conveniently, a body mass index (BMI) is calculated, where a person having a BMI greater than 25 is overweight and may be considered for treatment with the subject peptides. GPR39 directed agents find use in promoting gastric stasis and anorexic behavior without concomitant activation of the ACTH-glucocorticoid axis.

In a related embodiment, the treatment of non-insulin-dependent diabetes mellitus (NIDDM) is closely related to the treatment of obesity. NIDDM is a metabolic disease that affects about 5% to 7% of the population in western countries (and 10% of individuals over age 70). It is characterized by hyperglycemia and often accompanied by a number of other conditions, including hypertension, obesity and lipid disturbances. Patients are generally categorized as diabetic or hyperglycemic by measuring the level of glucose in the blood, either directly or by monitoring the level of glycosylated hemoglobin. Treatment is recommended where fasting glucose levels are greater 140 mg/dl, where bedtime glucose is greater than 160 mg/dl, or where HbA_{1c} is greater than 8%. The level of reduction that is desirable depends on the condition of the patient, and the blood glucose levels at the start of treatment, but generally about a 10 to 40% reduction in blood glucose is desirable, usually about a 25 to 35% reduction.

Hypertension is a disease which, if untreated, strongly predisposes to atherosclerotic cardiovascular disease. It is estimated that as many as 1 in 4 adult Americans have hypertension. Hypertension is approximately twice as common in persons with diabetes as in those without. The prevalence of hypertension increases with age.

Hypertension should not be diagnosed on the basis of a single measurement. Initial elevated readings should be confirmed on at least two subsequent visits over one week or more with average diastolic blood pressure of 90 mmHg or greater or systolic blood pressure of 140 mmHg or greater required for diagnosis of hypertension. Special care is warranted in diagnosing hypertension in persons with diabetes because of greater variability of blood pressure and a much greater likelihood of isolated systolic hypertension. A goal blood pressure of less than 130/85 mmHg is recommended for these patients.

In addition to dietary changes, pharmacological treatment may be required to control high blood pressure. The subject peptides may be administered to reduce arterial blood pressure. In addition, a secondary effect of reducing hypertension is reduction of edema and inflammatory exudate volume.

Pharmaceutical compositions containing GPR39 directed agents are useful as cardioprotective agents, e.g. to ameliorate ischemic injury or myocardial infarct size consequent to myocardial ischemia. The development of new therapeutic agents capable of limiting the extent of myocardial injury, i.e., the extent of myocardial infarction, following acute myocardial ischemia is a major concern of modern cardiology. There has also been interest in the development of therapies capable of providing additional myocardial protection which could be administered in conjunction with thrombolytic therapy, or alone, since retrospective epidemiological studies have shown that mortality during the first few years following infarction appears to be related to original infarct size.

Myocardial ischemia is the result of an imbalance of myocardial oxygen supply and demand and includes exertional and vasospastic myocardial dysfunction. Exertional ischemia is generally ascribed to the presence of critical atherosclerotic stenosis involving large coronary arteries resulting in a reduction in subendocardial flow. Vasospastic ischemia is associated with a spasm of focal variety, whose onset is not associated with exertion or stress. The spasm is better defined as an abrupt increase in vascular tone.

The compounds of this invention can be normally administered orally or parenterally, in the treatment of patients in need of cardioprotective therapy. The dosage regimen is that which insures maximum therapeutic response until improvement is obtained and thereafter the minimum effective level that gives relief. Thus, in general, the dosages are those that are therapeutically effective in producing a cardioprotective effect, i.e., amelioration of ischemic injury or myocardial infarct size consequent to myocardial ischemia. It is also anticipated that the peptides would be useful as an injectable dosage form, which may be administered in an emergency to a patient suffering from myocardial ischemia, etc.

In one aspect, the invention features a method of beneficially regulating gastrointestinal motility in a subject by administering to said subject a therapeutically effective amount of a GPR39 directed agent. In one embodiment, the methods of the present invention are directed to reducing gastric motility. In another embodiment, the invention is directed to methods of delaying gastric emptying. These methods may be used on a subject undergoing a gastrointestinal diagnostic procedure, for example radiological examination or magnetic resonance imaging. Alternatively, these methods may be used to reduce gastric motility in a subject suffering from a gastrointestinal disorder, for example, spasm (which may be associated with acute diverticulitis, a disorder of the biliary tract or a disorder of the Sphincter of Oddi). In another aspect, the invention is directed to a method of treating post-prandial dumping syndrome in a subject by administering to the subject a therapeutically effective amount of a GPR39 directed agent. In another aspect, the invention is directed to a method of treating post-prandial hyperglycemia by administering to a subject a therapeutically effective amount of a GPR39 agonist, e.g. post-prandial hyperglycemia as a consequence of Type 2 diabetes mellitus.

In another aspect, the present invention is directed to a method of treating gastric hypomotility in a subject by administering to the subject a therapeutically effective amount of a GPR39 antagonist. These methods may be employed where hypomotility is a consequence of diabetic neuropathy or where hypomotility is a consequence of anorexia nervosa. Hypomotility may also occur as a consequence of achlorhydria or as a consequence of gastric surgery. In another aspect, the invention is directed to a method of accelerating gastric emptying in a subject by administering to the subject a therapeutically effective amount of an obestatin modulator.

### Diagnostic and Prognostic Methods

Alterations in the genetic sequence of GPR39 may be associated with a predisposition to obesity and other conditions mediated by GPR39. In one embodiment, the alteration in the genetic sequence is an alteration at position 50 of the amino acid sequence, in the transmembrane region I of GPR39, changing an alanine to valine. In other embodiments, GPR39 polymorphisms are identified by methods known in the art, for example screening known databases of human genetic sequences, and the like.

A variety of different assays can be utilized to detect genetic alterations, including both methods that detect gene transcript and proteins. More specifically, the diagnostic and prognostic methods disclosed herein involve obtaining a sample from an individual and determining at least qualitatively the presence of polymorphisms as compared to the wild-type GPR39 sequence.

Nucleic acids or binding members such as antibodies that are specific for polypeptides derived GPR39 are used to screen patient samples for polymorphisms of the corresponding mRNA or protein, or for the presence of amplified DNA in the cell. Samples can be obtained from a variety of sources. Samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from spinal fluid, or tumor biopsy samples. Also included in the term are derivatives and fractions of such cells and fluids. Samples can also be derived from in vitro cell cultures, including the growth medium, recombinant cells and cell components. Diagnostic samples are collected from an individual that has, or is suspected to have, a brain tumor. The presence of specific markers is useful in identifying and staging the tumor.

Nucleic Acid Screening Methods

Some of the diagnostic and prognostic methods that involve the detection of a GPR39 genetic sequence begin with the lysis of cells and subsequent purification of nucleic acids from other cellular material, particularly DNA, and mRNA transcripts. A number of methods are available for analyzing nucleic acids for the presence of a specific sequence. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp. 14.2-14.33.

A detectable label may be included in an amplification reaction. Suitable labels include fluorochromes, e.g. ALEXA dyes (available from Molecular Probes, Inc.); fluorescein isothiocyanate (FITC), rhodamine, Texas Red, phycoerythrin, allophycocyanin, 6-carboxyfluorescein (6-FAM), 2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N, N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution. A solution containing labeled probes is then contacted with the cells and the probes allowed to hybridize. Excess probe is digested, washed away and the amount of hybridized probe measured. This approach is described in greater detail by Nucleic Acid Hybridization: A Practical Approach (Hames, et al., eds., 1987).

A variety of so-called “real time amplification” methods or “real time quantitative PCR” methods can also be utilized to determine the quantity of mRNA present in a sample. Such methods involve measuring the amount of amplification product formed during an amplification process. Fluorogenic nuclease assays are one specific example of a real time quantitation method that can be used to detect and quantitate transcripts. In general such assays continuously measure PCR product accumulation using a dual-labeled fluorogenic oligonucleotide probe—an approach frequently referred to in the literature simply as the “TaqMan” method. Additional details regarding the theory and operation of fluorogenic methods for making real time determinations of the concentration of amplification products are described, for example, in U.S. Pat. No. 5,210,015 to Gelfand, U.S. Pat. No. 5,538,848 to Livak, et al., and U.S. Pat. No. 5,863,736 to Haaland, each of which is incorporated by reference in its entirety.

Polypeptide Screening Methods

Screening may be based on the functional or antigenic characteristics of the protein. Various immunoassays designed to detect polymorphisms in GPR39 polypeptides. Detection may utilize staining of cells or histological sections, performed in accordance with conventional methods, using antibodies or other specific binding members. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the polypeptide in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding. After incubation, the insoluble support is generally washed of non-bound components. After washing, a solution containing a second antibody is applied. The antibody will bind to one of the proteins of interest with sufficient specificity such that it can be distinguished from other components present. The second antibodies may be labeled to facilitate direct, or indirect quantification of binding. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the targeted polypeptide, conveniently using a labeling method as described for the sandwich assay.

In some cases, a competitive assay will be used. In addition to the patient sample, a competitor to the targeted protein is added to the reaction mix. The competitor and the target compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

### Example 1

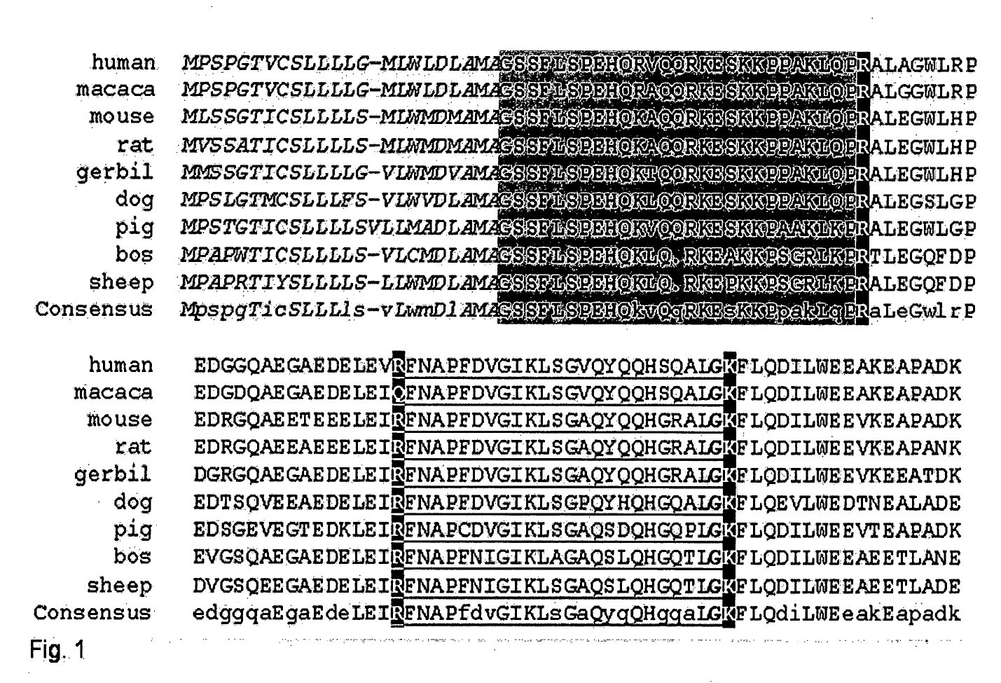

GenBank was searched for orthologs of the human ghrelin gene in diverse vertebrates, and preproghrelin sequences were compared from several mammalian species to reveal conserved regions. In addition to the known ghrelin mature peptide (FIG. 1, shaded) immediately following the signal peptide, another conserved region (underlined) flanked by potential convertase cleavage sites was identified. This amidated peptide with 23 amino acid residues was named as ghrelin associated peptide (GLAP, obestatin).

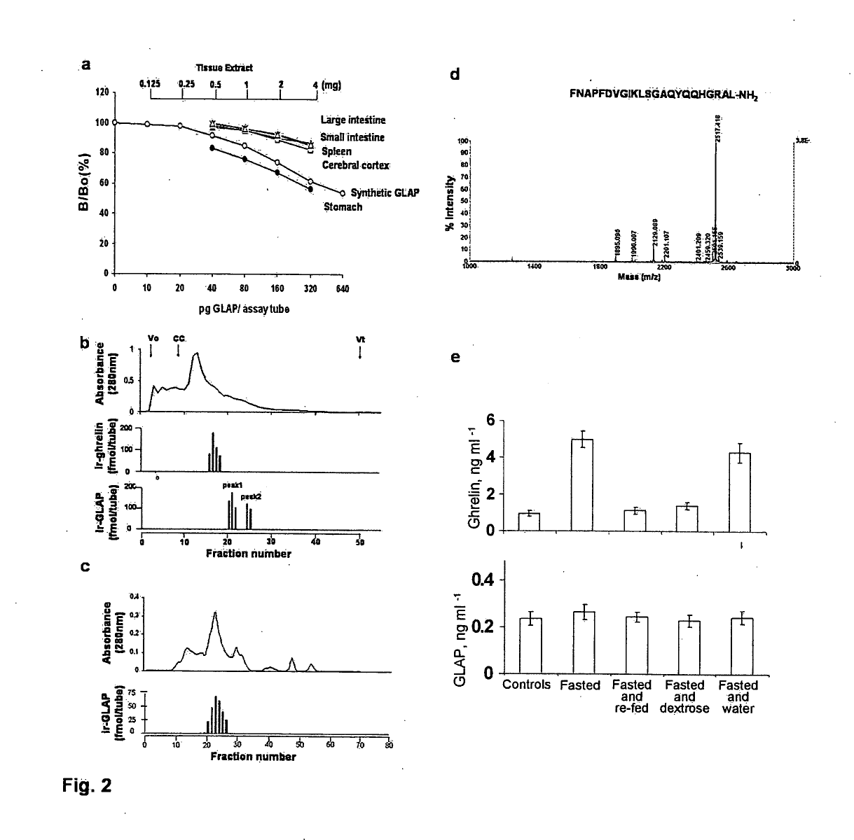

To detect endogenous obestatin, we extracted diverse rat tissues and performed radioimmunoassays using specific obestatin antibodies generated against the synthetic peptide. As shown in FIG. 2a, the stomach extract displaced labeled obestatin binding to the obestatin antibodies in a dose-dependent manner whereas several other tissues showed minimal competition.

Obestatin-like activities from stomach extracts of 30 rats were purified. As shown in FIG. 2b, immunoreactive obestatin was eluted in a Sephadex G-50 gel permeation column with estimated sizes of 2.6 and 1.5 kDa, distinct from the elution position of mature ghrelin. Because peak 1 corresponded to the predicted size of obestatin, we subjected these fractions to ion-exchange FPLC.

As shown in FIG. 2c, a single peak of immunoreactive obestatin could be eluted. Mass spectrometry and Edman sequencing analyses showed that this peak contained a peptide with a molecular mass of 2516.3 (FIG. 2d) and the sequence of FNAPFDVGIKLSGAQYQQHG-XX. Combined with molecular weight determination, the full sequence of the purified peptide was predicted to be FNAPFDVGIKLSGAQYQQHGRAL-NH₂, consistent with the obestatin sequence deduced from rat ghrelin cDNA. In addition, mass spectrometric analyses suggested that peak 2 represented the last 13 residue of amidated obestatin. To investigate differential secretion of ghrelin and obestatin releases in vivo, adult male rats were fasted for 48 h before re-feeding. Consistent with earlier findings, fasting led to a major increase in serum ghrelin levels whereas free access to food or drinking water containing dextrose for two hours decreased circulating ghrelin (FIG. 2e). In contrast, serum obestatin showed no changes in different treatment groups.

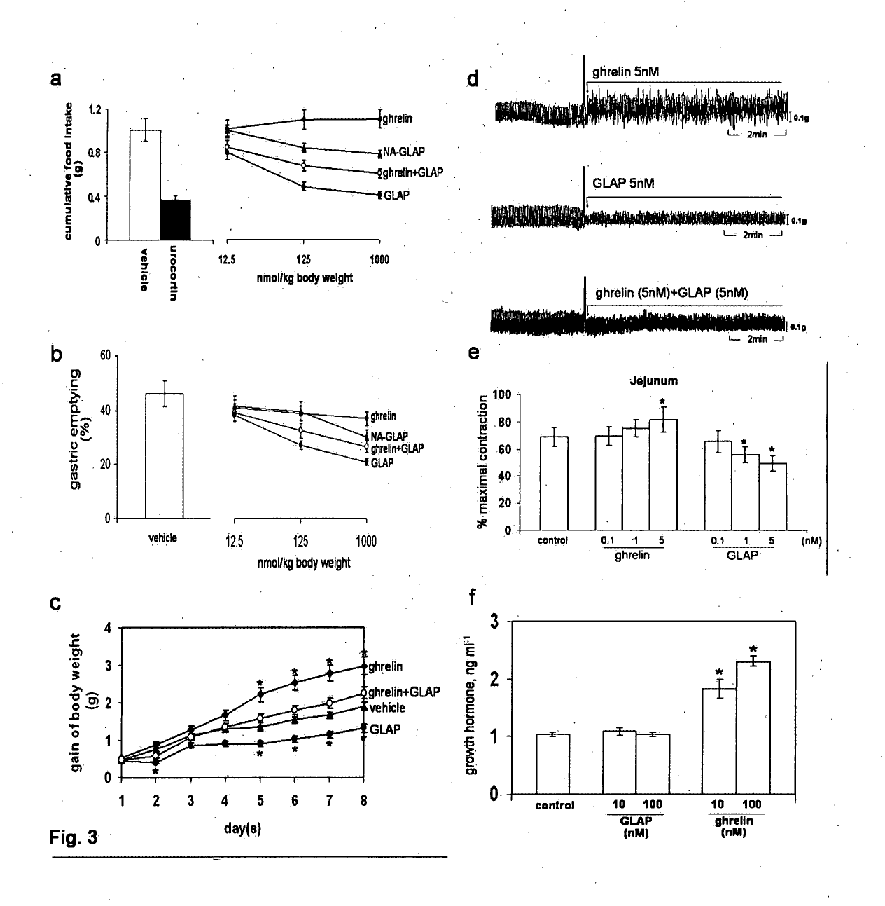

We synthesized amidated human obestatin to test its biological actions. Adult male mice were treated intraperitoneally with obestatin before measurement of food intake and gastric emptying activities. As shown in FIG. 3a, treatment with obestatin, like urocortin, suppressed food intake for five hours in a dose-dependent manner. However, treatment with ghrelin or the non-amidated obestatin (NA-obestatin) was minimally effective. Treatment with obestatin also led to a sustained suppression of gastric emptying activity (FIG. 3b) in a dose-dependent manner. However, treatment with NA-obestatin was less effective and treatment with ghrelin was ineffective. Based on the reported orexigenic effects of ghrelin, adult male rats were injected with ghrelin, obestatin, or vehicle alone. As shown in FIG. 3c, treatment with ghrelin (1 μmol/kg body weight, thrice daily) increased body weight but treatment with the same dose of obestatin suppressed body weight gain. Furthermore, cotreatment with obestatin antagonized the orexigenic action of ghrelin. As shown in FIGS. 3d and e, isometric force measurement demonstrated that treatment with obestatin decreased contractile activity of jejunum muscle strips and antagonized the known stimulatory effect of ghrelin. FIG. 3f shows that unlike ghrelin, treatment of cultured rat pituitary cells with obestatin did not increase GH secretion (ng/culture: control, 1.04+/−0.03; obestatin (100 nM)-treated, 1.04+/−0.03; ghrelin (100 nM)-treated, 2.32+/−0.08).

To study obestatin receptors, binding of I¹²⁵-obestatin to the crude plasma membrane preparation of rat jejunum was determined. Labeled obestatin interacted with jejunum preparations with a high affinity (Kd: 4 nM). Furthermore, obestatin binding to its target cells was not competed by ghrelin, motilin, neurotensin, and neuromedin U. Also, NA-obestatin and the truncated (des1-10)obestatin were less effective than obestatin. In addition to jejunum, specific I¹²⁵-obestatin binding sites were also found in the pituitary, stomach, and ileum, but lower in other tissues. To test if obestatin interacted with a G protein-coupled receptor (GPCR), a non-hydrolyzable GTP analog GTPγS was added to the binding assay. Incubation with GTPγS led to a dose-dependent suppression of I¹²⁵-obestatin binding to the jejunum preparation with 5, 20, and 45% inhibition at 1, 10, and 100 uM, respectively. These findings suggest that obestatin could bind a GPCR.

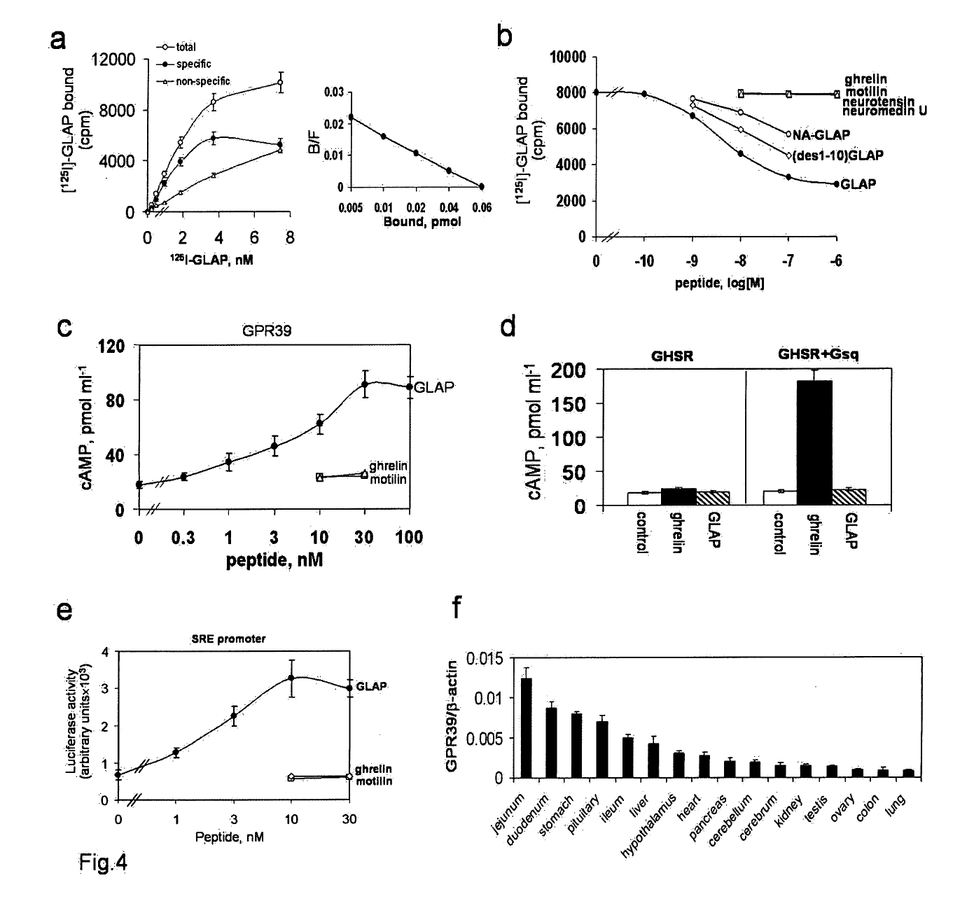

We hypothesized that obestatin interacts with a GPCR belonging to the ghrelin receptor family and tested the binding of obestatin to these receptors. As shown in FIG. 4a, I¹²⁵-obestatin interacted with the orphan receptor GPR39 with a high affinity (Kd: 1 nM). Furthermore, obestatin binding to GPR39 was competed by obestatin but not by ghrelin, motilin, neurotensin, and neuromedin U (FIG. 4b). In addition, NA-obestatin and truncated (des1-10)obestatin were less effective. In CHO cells overexpressing GPR39, treatment with obestatin led to dose-dependent increases in cAMP production whereas treatments with ghrelin or motilin were ineffective (FIG. 4c). Although CHO cells expressing GHSR did not respond to treatment with obestatin or ghrelin, cotransfection with a chimeric Gsq protein led to cAMP increases induced by ghrelin but not obestatin (FIG. 4d). In addition, cotransfection of both GPR39 and a SRE promoter-luciferase construct conferred obestatin but not ghrelin or motilin signaling (FIG. 4e). Real-time reverse-transcription PCR analyses further indicated that GPR39 is expressed in jejunum, duodenum, stomach, pituitary, ileum, liver, and other tissues, consistent with obestatin binding studies.

Combining bioinformatic analyses of evolutionarily conserved regions in the preproghrelin sequences of diverse mammalian species, followed by experimental verification, we identified obestatin as a novel polypeptide hormone. This amidated peptide could be purified from the rat stomach, interacts with the orphan GPR39 expressed in gastrointestinal, pituitary and other tissues. In direct contrast to the orexegenic actions of acylated ghrelin, amidated obestatin showed anorexic effects in regulating gastrointestinal functions.

Our findings of two peptide hormones derived from the same proprotein but acting thorough distinct receptors to exert opposing physiological actions indicate the existence of important regulatory mechanisms at post-translational levels. Thus, monitoring of ghrelin transcript levels is inadequate to reflect the secretion of these two polypeptides with diametrically opposing gastrointestinal functions. After removal of the signal peptides from pre-proproteins of regulatory peptides, prohormone convertases cleave prohormones at mono- or dibasic residues. For processed peptides with a C-terminal glycine, this residue is further converts to NH2 by the amidation enzyme PAM, producing an amidated peptide. Similar to the importance of the posttranslational amidation for obestatin bioactivity, the mature ghrelin peptide also requires n-octanoylation on its serine (3) residue by an uncharacterized acyl transferase enzyme for its biological activity. Thus, the derivation of these two peptides from the same precursor is likely regulated at the posttranslational level.

Ghrelin is implicated in meal initiation, and body weight regulation. Chronic ghrelin administration increases food intake and decreases energy expenditure, causing body weight gain. Unlike ghrelin which causes hyperphagia and obesity in rats, obestatin acts as an anorexic hormone, by decreasing food intake, gastric emptying activities, jejunum motility, and body weight gain. Of interest, mutant mice with a deletion of the ghrelin gene did not show impaired growth or appetite, likely because these animals lacked both orexigenic ghrelin and anorexic obestatin. Indeed, transgenic mice bearing the preproghrelin gene under the control of the chicken β-actin promoter produced high levels of inactive des-acyl ghrelin but exhibited lower body weights, likely due to the biosynthesis of obestatin.

The present discovery of a novel polypeptide hormone and its cognate receptor underscore the power of comparative genomic analyses in the postgenomic era. From nine mammalian species examined, the deduced sequences of obestatin, their flanking proteolytic cleavage sites, and the amidation donor glycine residue are highly conserved. The prediction of an amidated obestatin was confirmed following purification of endogenous obestatin from the rat stomach. Receptor binding and biological assays also demonstrated that the amidated form is much more potent than its non-amidated counterpart. Although another conserved lysine (10) residue was found in obestatin and proteolytic cleavage at this site could lead to a truncated obestatin peptide similar to peak 2 found in FIG. 2b, synthetic des(1-10)Obestatin was less potent than obestatin in the receptor binding assay, suggesting the 23 residue obestatin is the predominant bioactive peptide.

There are many examples of one prohormone giving raise to more than one mature polypeptide hormones. For hormones originated from orexin and tachykinin genes (REF), different peptides derived from the same parent proprotein show sequence homology and interact with one or more paralogous receptors with overlapping specificity. For hormones derived from the parent proopiomelanocortin (MSH and ACTH) and proglucagon (glucagon, GLP1, and GLP2), many of the mature peptides interact with receptors with overlapping ligand specificity, distinct tissue distribution, and diverse biological roles. Ghrelin and obestatin provide an extreme example of a precursor protein giving rise to mature peptides without sequence homology and interact with distinct receptors to mediate disparate functions.

Several polypeptides are derived from proghrelin. In addition to the n-octanoylated ghrelin, des-Gln(14)-ghrelin and des-Arg(28) were purified from the stomach of rat and man, respectively. Furthermore, a peptide derived from the 66 C-terminal amino acids of proghrelin, named as C-ghrelin, was detected in the human circulation and its serum levels were significantly elevated in patients with heart failure. Although the antibodies used to detect c-ghrelin overlap with obestatin by 13 residues, the exact chemical nature and function of the circulating peptides remain to be established.

Of interest, an alternatively spliced ghrelin variant with the deletion of exon 4 encoding obestatin has been reported (Jeffery, Duncan et al. 2005). In addition, at least two alternatively spliced ghrelin transcripts have been found in mice. The ghrelin gene derived transcript (GGDT) found in the testis encodes obestatin, but not mature ghrelin (Tanaka, Hayashida et al. 2001) whereas the exon 4-deleted variant found in mouse encodes ghrelin but not obestatin (Jeffery et al. (2005) Endocrinology 146(1): 432-40). However, the physiological significance of the testis-specific transcript is unclear because the predicted protein sequence does not contain a signal peptide.

Ghrelin binds to GHSR belonging to the subgroup of type A GPCRs consisting of motilin receptor/GPR38, two neurotensin receptors, two neuromedin U receptors, the TRH receptor, and the orphan receptor GPR39 (Smith et al. (1999) Horm Res 51 Suppl 3:1-8). Our discovery that obestatin is the cognate ligand for GPR39 suggests that GHSR and GPR39 could have evolved from a common ancestor but diverged in their functions to maintain a delicate balance of body weight regulation. This scenario is similar to the divergent and sometimes opposing actions of the paralogous CRH receptors 1 and 2 activated by several paralogous ligands to maintain the delicate adaptive stress responses (Hsu and Hsuch 2001; Lewis, Li et al. 2001).

In addition to its roles in meal initiation, body weight regulation and gastrointestinal activity, ghrelin also regulates pituitary hormone axis, carbohydrate metabolism, and functions of heart, kidney, pancreas, adipose tissues, and gonads. Because ghrelin mRNA was found in almost all human tissues analyzed, the present identification of obestatin derived from the same gene product as ghrelin provides the basis for future elucidation of the differential posttranslational processing and modification of these two peptides. It is important to investigate the release of ghrelin and obestatin under different physiological states and the molecular mechanisms underlying their activation of distinct receptors to allow a better understanding of the intricate balance of energy homeostasis, body weight control, and related physiological processes.

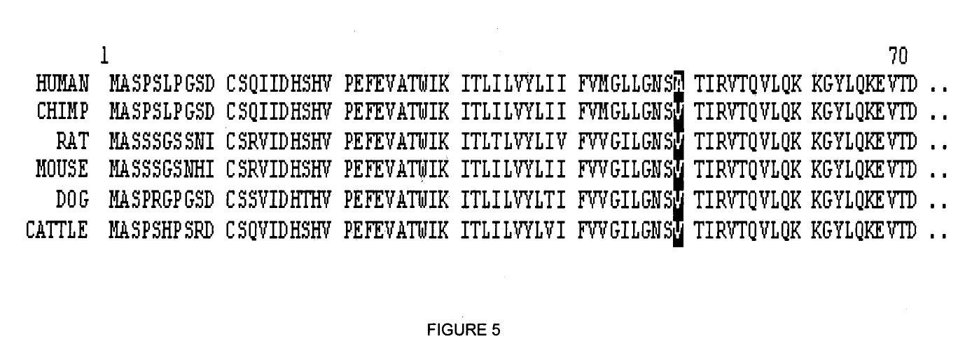

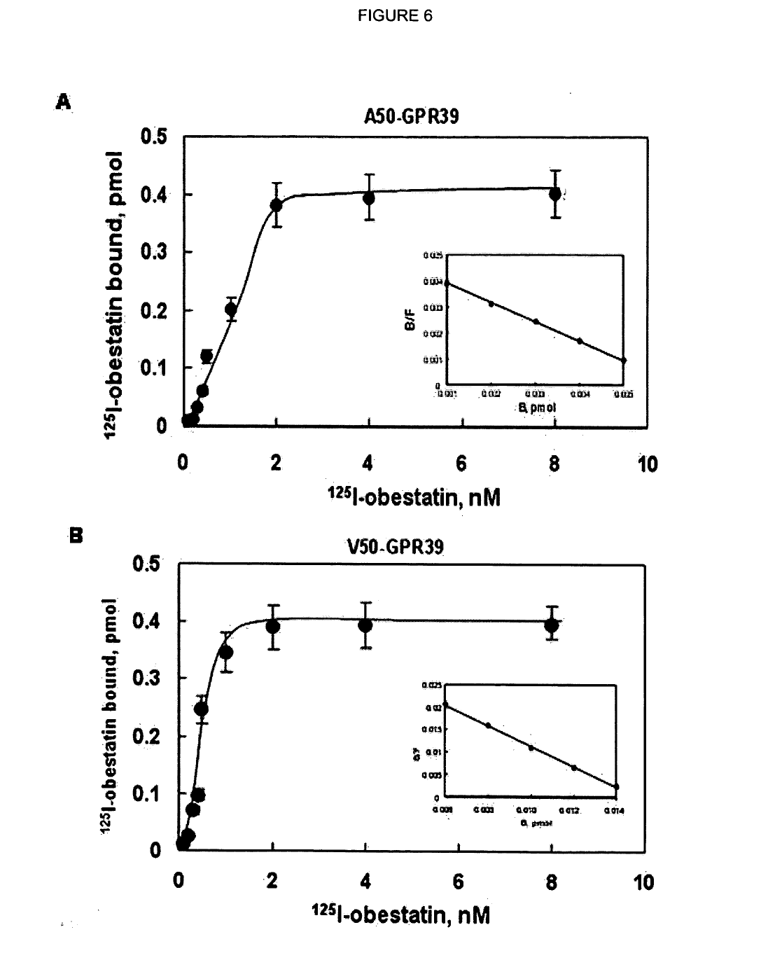

GPR39 has a 149G->A polymorphism that changes Ala 50 to Val in the first transmembrane region. The estimated SNP heterozygosity for GPR39 is 0.413, with major and minor allele frequencies of 0.709 and 0.291, respectively, from a study of 331 people of seven populations. The L15-obestatin variant has been synthesized and site-directed mutagenesis was performed to generate the A50V GPR39 variant. Saturation binding of iodinated wild type obestatin to CHO cells transfected with wild type GPR39 or the 50V-GPR39 variant was performed. As shown in FIG. 6, the V50-GPR39 receptor variant exhibited a two-fold higher affinity to obestatin than the wild type A50-GPR39 (Kd values: A50-GPR39: 1.15 nM; V50-GPR39: 0.45 nM). Of interest, the valine residue in position 50 is conserved in all mammalian species examined (FIG. 5), suggesting the importance of this valine residue in receptor function. These findings indicate the V50-GPR39 isoform could allow more efficient obestatin ligand signaling.

Methods

Reagents. Human obestatin, non-amidated obestatin (NA-obestatin) and (des1-10)obestatin were synthesized by GL Biochem Ltd. (Shanghai, China) and purified by reversed phase high-performance liquid chromatography. The peptide sequences were verified by amino acid analysis and mass spectrometry. Ghrelin, motilin, neurotensin, neuromedin U, and anti-proghrelin IgG were purchased from Phoenix Pharmaceuticals (Belmont, Calif.). The rabbit polyclonal antibodies against synthetic human obestatin peptide were produced by Quality Controlled Biochemicals (Hopkinton, Mass.).

Radioimmunoassay of obestatin and ghrelin. Tissue extracts were used for the measurement of obestatin and ghrelin immunoreactivities. Immuno-obestatin was determined using rabbit polyclonal antibodies at a final dilution of 1:6,000. The reaction mixture consisted of 100 μl of test samples, or standards together with 100 μl of the antiserum. Tubes were incubated for 24 h at 4 C before adding 100 μl of tracer (10,000-15,000 cpm) for further incubation for 24 h at 4 C. Free and bound obestatin was separated by the solid phase second antibody method using donkey anti-rabbit IgG (Phoenix Pharmaceuticals) before counting in a γ-spectrometer (LKB, Uppsala, Sweden). Total (n-octanoyl and des-acyl) ghrelin immunoreactivity was determined using a specific radioimmunoassay (Phoenix Pharmaceuticals). No cross-reactivity was found between the obestatin and ghrelin radioimmunoassays. Circulating ghrelin and obestatin levels were measured in adult male Sprague-Dawley rats (n=5 per group) before and after fasting for 48 h, or fasting followed by free access to food or drinking water containing 50% dextrose for 2 h.

Purification of obestatin. Stomach preparations (67 g) from 30 rats were minced and boiled for 5 min. in 5 volumes of water to inactivate intrinsic proteases. Before homogenization with a Polytron mixer, the solution was adjusted to 1 M acetic acid and 20 mM HCl. After centrifugation at 225,000 g for 30 min., the supernatant was concentrated to 100 ml using an evaporator before precipitation under 66% acetone. After removing the precipitates, the volume of the supernatant was reduced by acetone evaporation before loading onto a 10-g cartridge of Sep-Pak C18 (Waters), pre-equilibrated with 0.1% trifluoroacetic acid (TFA). The Sep-Pak cartridge was washed with 10% acetonitrile/0.1% TFA, and then eluted with 60% acetonitrile/0.1% TFA. Peptides in the eluate was lyophilized, dissolved in 1 M acetic acid, and fractionated using a Sephadex G-50 gel-filtration column. A portion of each fraction was used for obestatin and ghrelin radioimmunoassays. Fractions containing immuno-obestatin were further separated by ion-exchange FPLC on a UNO Q1 column (Biorad) at pH 8.1. After identifying the peak containing immuno-obestatin, the samples were subjected to mass spectrometry and de novo N-terminal sequencing (Pan Facility, Stanford University, Calif.).

Analysis of gastrointestinal functions. Eight-week-old C57BL6 male mice were housed individually in a regulated environment. Before intraperitoneal treatment with different peptides, mice were deprived of food for 16 h with free access to water. Food intake was measured by placing preweighed pellets in the cage and weighing uneaten pellets at 1, 3 and 5 h after treatment. To estimate gastric emptying responses, mice deprived of food for 16 h were given food pellets for 90 min. before injection of different hormones or saline. After treatment, mice were deprived of food again and killed 2 h later. The stomach was excised at the pylorus and cardia before weighing. Gastric emptying was calculated by subtracting the stomach weight of treated mice from those killed at the time of the hormone injection.

Isometric force measurements. Jejunum muscle strips (˜1 cm in length) were cut along the longitudinal axis of the circular muscle layer, and the mucosa was removed to minimize endogenous peptides. Muscle strips were mounted to a TIS8105R (Kent Scientific Corporation, Torrington, Conn.) isometric strain gauge and immersed in a 5 ml organ bath maintained at 37° C. with oxygenated KRB [Krebs-Ringer phosphate buffer, consisting of 50 mM HEPES, 100 mM NaCl, 5 mM KCl, and 1 mM each of MgCl2, NaH2PO4, and CaCl2] as described earlier (Porcher et al. (2005) Am J Physiol Gastrointest Liver Physiol 288(5): G1091-103.). A resting force of 1.0 g was applied to intestinal muscles and a 1.5 h equilibration period was allowed before testing of different peptides during a 5 min. period by incubating muscles with peptides at desired concentrations. After each test, muscle strips were washed with fresh KRB for 20 min. to allow full recovery of basal contractile activities. Maximal contraction was evaluated at the beginning and end of each experiment following treatment with 10 μM of acetylcholine chloride. Contractility data were digitized and stored in a computer using the Acknowledge software (MP 100, Biopac Systems, Inc., Goleta, Calif.). Magnitude of contractile strength is expressed as % of maximal contraction induced by acetylcholine.

Pituitary cell cultures. Anterior pituitaries were removed from adult male Sprague-Dawley rats and dispersed with 0.3% collagenase, 0.1% hyaluronidase and DNase I (10 μg/ml). The cell suspension was centrifuged at 300 g for 5 min. and the cells were washed twice before incubated in DMEM containing 0.1% bovine serum albumin (BSA), penicillin (100 units/ml), streptomycin (100 μg/ml), fungizone (2.5 μg/ml) and 10% fetal calf serum. Cells were seeded onto poly-lysine-coated 24-well plates at a density of 2˜3×10⁵ cells/well. Cultures were kept in a humidified atmosphere of 5% CO₂ and 95% air at 37° C. After a 72 h-incubation, the cells were washed with DMEM containing 0.1% BSA, then incubated for 1 h in serum-free medium with ghrelin or obestatin. Media was removed and rat GH was measured by an ELISA.

Labeling of obestatin and receptor binding. Iodination of obestatin was performed using the Iodogen (Pierce, Upland, Ind.) procedure. Mixture of the peptide (20 μg) and 1 mCi [¹²⁵I] NaI was transferred to precoated Iodogen vials and incubated for 4 min. The ¹²⁵I-labeled peptide was applied to a Sep-Pak C18 cartridge (Waters, Milford, Mass.) before elution with 60% acetonitrile/0.1% TFA. For radioligand binding assays, rat jejunum or other tissues were washed with buffer A (20 mM Hepes, 5 mM EDTA, 1 mM dithiothreitol (DTT), 10 μM amidinophenylmethanesulfonyl fluoride, 5 mg/L leupeptin, 100 mM KCl, pH 7.5), cut into small pieces and homogenized using a Physcotron motorized homogenizer. The homogenates were centrifuged at 1,000 g for 5 min. and the supernatant was centrifuged at 300,000 g for 1 h at 2° C. The pellets (crude membrane fractions) were resuspended with buffer A without KCl, quickly frozen under liquid nitrogen, and stored at −80° C. until use. Tissue homogenates were incubated in 100 μl of PBS containing 0.1% bovine serum albumin for 18 h at room temperature with varying concentrations of ¹²⁵I-obestatin in the presence or absence of unlabelled obestatin at 1,000-fold excess. After incubation, the tubes were centrifuged for 10 min. at 10,000 g, and the pellet was washed twice in ice-cold PBS before counting using a γ-spectrophotometer. Specific binding was calculated by subtracting nonspecific binding from total binding. For displacement curves, a fixed concentration of ¹²⁵I-obestatin was incubated with or without increasing concentrations of obestatin or other peptides.

Quantitative real-time RT-PCR. To quantify transcript levels for GPR39 in mouse tissues, real-time RT-PCR was performed using a SmartCycler (Cepheid, Sunnyvale, Calif.) as described previously (Luo, Dewey et al. 2005). Total RNA was extracted from different mouse tissues using the RNeasy kit (Qiagen Science, Valencia, Calif.) before reverse transcription using a Sensiscript RT kit (Qiagen Science). Real-time PCR was performed using a QuantiTect Probe PCR Kit (Qiagen Sciences) and fluorescent-labeled probes (3′-end: TAMRA, 5′-end: 6-FAM). Expression of β-actin was used for copy number normalization. Standard curves for GPR39 and β-actin transcripts were generated by serial dilutions of individual cDNAs. The primer pairs and fluorescent probes used were as follows: GPR39 forward: 5′-AGACAGACCATCATATTCCTGAGAC-3′; GPR39 reverse: 5′-AGTACGTTCTGGTCCAGTCATGTT-3′; GPR39 probe: 5′-FAM-TGCCCAATCAGATCCGACGGATCA-TAMRA-3′; β-actin forward: 5′-TCTGTGTGGATTGGtGGCTCTA-3′; β-actin reverse: 5′-CTGCTTGCTGATCCACATCTG-3′; β-actin probe: 5′-FAM-CTTGCCCACAGCCTTGGCAGC-TAMRA-3′.

Statistical analysis. Differences between treatment groups were analyzed using ANOVA and the Student's t-test.

All publications and patent applications cited in this specification are herein incorporated by reference as if each individual publication or patent application were specifically and individually indicated to be incorporated by reference. The publications discussed herein are provided solely for their disclosure prior to the filing date of the present application. Nothing herein is to be construed as an admission that the invention is not entitled to antedate such a disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of screening for biologically active agents that modulate obestatin function, the method comprising:
combining a candidate biologically active agent with a GPR39 polypeptide or a cell expressing GPR39; and
determining the effect of said agent on obestatin function.

2. The method according to claim 1, wherein said method comprises determining binding of said candidate biologically active agent to said GPR39 polypeptide.

3. The method according to claim 1, wherein said method comprises determining the effect of said candidate biologically active agent on GPR39 mediated signaling activity in a cell.

4. The method according to claim 1, comprising the step of comparing the effect of said candidate biologically active agent with the effect of obestatin.

5. The method according to claim 1, wherein obestatin is used in a competition assay with said candidate biologically active agent.

6. The method according to claim 1, wherein said candidate biologically active agent is a small organic molecule.

7. The method according to claim 1, wherein said candidate biologically active agent is a polypeptide.

8. The method according to claim 7, wherein said polypeptide is derived from obestatin.

9. The method according to claim 1, wherein said candidate biologically active molecule is an antibody or fragment thereof.

10. A method for the treatment of obesity in a mammalian subject, the method comprising:
administering to said subject an effective dose of a ligand of GPR39.

11. A method for the regulation of blood pressure in a mammalian subject, the method comprising:
administering to said subject an effective dose of a ligand of GPR39.

12. A method for the regulation of gut motility, the method comprising:
administering to said subject an effective dose of a ligand of GPR39.

13. A method of screening for a predisposition to obesity, the method comprising:
contacting a patient sample comprising a GPR39 sequence; and
determining the presence of a GPR39 polymorphism associated with obesity.
